(19) United States
(12) Patent Application Publication
(10) Pub. No.: US 2024/0245628 A1
(43) Pub. Date: Jul. 25, 2024

(54) COMPOSITIONS AND METHODS FOR HEALTH COMPRISING CO-ENZYME Q10

(71) Applicant: Bausch + Lomb Ireland Limited, Dublin (IE)

(72) Inventors: Daniel STEIN, Bridgewater, NJ (US); Christopher CORTINA, Dublin (IE)

(21) Appl. No.: 18/402,046

(22) Filed: Jan. 2, 2024

Related U.S. Application Data
(60) Provisional application No. 63/478,306, filed on Jan. 3, 2023.

(51) Int. Cl. A61K 31/122; A61K 31/05; A61K 31/355; A61K 31/375; A61K 33/30; A61K 33/34; A61K 45/06

(52) U.S. Cl. CPC A61K 31/122; A61K 31/05; A61K 31/355; A61K 31/375; A61K 33/30; A61K 33/34; A61K 45/06

(57) ABSTRACT

The present disclosure describes compositions comprising vitamin C, vitamin E, zinc, copper, lutein, zeaxanthin, and coenzyme Q10, and pharmaceutically acceptable salts of any of the foregoing. The compositions are useful in methods for treating conditions associated with a loss of visual acuity including, for example, age-related macular degeneration as well as supporting heart health.

This application claims the benefit of priority to U.S. Provisional Patent Application No. 63/478,306, filed Jan. 3, 2023, which is hereby incorporated by reference in its entirety.

Compositions and methods for treating and/or preventing at least one disease, disorder, and/or condition associated with a loss of visual acuity including, for example, age-related macular degeneration, and/or at least one disease, disorder, and/or condition associated with heart disease are disclosed herein.

In some embodiments, disclosed herein are compositions comprising vitamin C, a prodrug thereof, and/or a pharmaceutically acceptable salt of any of the foregoing; vitamin E, a prodrug thereof, and/or a pharmaceutically acceptable salt of any of the foregoing; zinc, a prodrug thereof, and/or a pharmaceutically acceptable salt of any of the foregoing; copper, a prodrug thereof, and/or a pharmaceutically acceptable salt of any of the foregoing; lutein, a prodrug thereof, and/or a pharmaceutically acceptable salt of any of the foregoing; zeaxanthin, a prodrug thereof, and/or a pharmaceutically acceptable form of any of the foregoing; and coenzyme Q10, a prodrug thereof, and/or a pharmaceutically acceptable form of any of the foregoing. In some embodiments, coenzyme Q10 is in the form of ubiquinol. In some embodiments, coenzyme Q10 is in the form of ubiquinone.

Also disclosed herein are compositions comprising 350 mg to 800 mg of vitamin C or an equivalent amount of a prodrug, a pharmaceutically acceptable salt, and/or a pharmaceutically acceptable salt of a prodrug of vitamin C; 100 mg to 550 mg of vitamin E or an equivalent amount of a prodrug, a pharmaceutically acceptable salt, and/or a pharmaceutically acceptable salt of a prodrug of vitamin E; 20 mg to 90 mg of zinc or an equivalent amount of a prodrug, a pharmaceutically acceptable form, and/or a pharmaceutically acceptable salt of a prodrug of zinc; 1.5 mg to 2.5 mg of copper or an equivalent amount of a prodrug, a pharmaceutically acceptable form, and/or a pharmaceutically acceptable salt of a prodrug of copper; 5 mg to 50 mg of lutein or an equivalent amount of a prodrug, a pharmaceutically acceptable salt, and/or a pharmaceutically acceptable salt of a prodrug of lutein; 1 mg to 20 mg of zeaxanthin or an equivalent amount of a prodrug, a pharmaceutically acceptable salt, and/or a pharmaceutically acceptable salt of a prodrug of zeaxanthin; 100 mg to 400 mg of coenzyme Q10 or an equivalent amount of prodrug, a pharmaceutically acceptable salt, and/or a pharmaceutically acceptable salt of a prodrug of coenzyme Q10.

Also disclosed herein are compositions comprising 500.0 mg vitamin C, optionally in the form of ascorbic acid; 180.0 mg vitamin E, optionally in the form of dl-alpha tocopheryl acetate; 80.0 mg zinc, optionally in the form of zinc oxide; 2.0 mg copper, optionally in the form of copper oxide; 10.0 mg R′R′ lutein; 2.0 mg R′R′ zeaxanthin; and 100 mg coenzyme Q10.

Also disclosed herein are compositions comprising 500.0 mg vitamin C, optionally in the form of ascorbic acid; 180.0 mg vitamin E, optionally in the form of dl-alpha tocopheryl acetate; 25 mg zinc, optionally in the form of zinc oxide; 2.0 mg copper, optionally in the form of copper oxide; 10.0 mg R′R′ lutein; 2.0 mg R′R′ zeaxanthin; and 400 mg coenzyme Q10, optionally in the form of ubiquinol.

Also disclosed herein are compositions comprising 500.0 mg vitamin C, optionally in the form of ascorbic acid; 180.0 mg vitamin E, optionally in the form of dl-alpha tocopheryl acetate; 25 mg zinc, optionally in the form of zinc oxide; 2.0 mg copper, optionally in the form of copper oxide; 10.0 mg R′R′ lutein; 2.0 mg R′R′ zeaxanthin; and 100 mg coenzyme Q10, optionally in the form of ubiquinone.

Also disclosed herein are compositions comprising 500.0 mg vitamin C, optionally in the form of ascorbic acid; 180.0 mg vitamin E, optionally in the form of dl-alpha tocopheryl acetate; 80 mg zinc, optionally in the form of zinc oxide; 2.0 mg copper, optionally in the form of copper oxide; 10.0 mg R′R′ lutein; 2.0 mg R′R′ zeaxanthin; and 100 mg coenzyme Q10, optionally in the form of ubiquinol.

Also disclosed herein are compositions comprising 500.0 mg vitamin C, optionally in the form of ascorbic acid; 180.0 mg vitamin E, optionally in the form of dl-alpha tocopheryl acetate; 80 mg zinc, optionally in the form of zinc oxide; 2.0 mg copper, optionally in the form of copper oxide; 10.0 mg R′R′ lutein; 2.0 mg R′R′ zeaxanthin; and 400 mg coenzyme Q10, optionally in the form of ubiquinone.

Also disclosed herein are compositions comprising 500.0 mg vitamin C, optionally in the form of ascorbic acid; 180.0 mg vitamin E, optionally in the form of dl-alpha tocopheryl acetate; 80.0 mg zinc, optionally in the form of zinc oxide; 2.0 mg copper, optionally in the form of copper oxide; 10.0 mg R′R′ lutein; 2.0 mg R′R′ zeaxanthin; and 100 mg coenzyme Q10, optionally in the form of ubiquinol.

Also disclosed herein are compositions comprising 500.0 mg vitamin C, optionally in the form of ascorbic acid; 180.0 mg vitamin E, optionally in the form of dl-alpha tocopheryl acetate; 80.0 mg zinc, optionally in the form of zinc oxide; 2.0 mg copper, optionally in the form of copper oxide; 10.0 mg R′R′ lutein; 2.0 mg R′R′ zeaxanthin; and 100 mg coenzyme Q10, optionally in the form of ubiquinone.

Also disclosed herein are compositions comprising 500.0 mg vitamin C, optionally in the form of ascorbic acid; 180.0 mg vitamin E, optionally in the form of dl-alpha tocopheryl acetate; 25.0 mg zinc, optionally in the form of zinc oxide; 2.0 mg copper, optionally in the form of copper oxide; 10.0 mg R′R′ lutein; 2.0 mg R′R′ zeaxanthin; and 100 mg coenzyme Q10, optionally in the form of ubiquinol.

Also disclosed herein are compositions comprising 500.0 mg vitamin C, optionally in the form of ascorbic acid; 180.0 mg vitamin E, optionally in the form of dl-alpha tocopheryl acetate; 25.0 mg zinc, optionally in the form of zinc oxide; 2.0 mg copper, optionally in the form of copper oxide; 10.0 mg R′R′ lutein; 2.0 mg R′R′ zeaxanthin; and 100 mg coenzyme Q10, optionally in the form of ubiquinone.

Also disclosed herein are compositions comprising about 350 mg to about 800 mg of vitamin C or an equivalent amount of a prodrug, a pharmaceutically acceptable salt, and/or a pharmaceutically acceptable salt of a prodrug of vitamin C; about 100 mg to about 550 mg of vitamin E or an equivalent amount of a prodrug, a pharmaceutically acceptable salt, and/or a pharmaceutically acceptable salt of a prodrug of vitamin E; about 20 mg to about 90 mg of zinc or an equivalent amount of a prodrug, a pharmaceutically acceptable form, and/or a pharmaceutically acceptable salt of a prodrug of zinc; about 1.5 mg to about 2.5 mg of copper or an equivalent amount of a prodrug, a pharmaceutically acceptable form, and/or a pharmaceutically acceptable salt of a prodrug of copper; about 5 mg to about 50 mg of lutein or an equivalent amount of a prodrug, a pharmaceutically acceptable salt, and/or a pharmaceutically acceptable salt of a prodrug of lutein; about 1 mg to about 20 mg of zeaxanthin or an equivalent amount of a prodrug, a pharmaceutically acceptable salt, and/or a pharmaceutically acceptable salt of a prodrug of zeaxanthin; and about 20 mg to about 400 mg of coenzyme Q10.

Also disclosed herein are compositions comprising about 500 mg vitamin C, optionally in the form of ascorbic acid; about 400 mg vitamin E, optionally in the form of dl-alpha tocopheryl acetate; about 80 mg zinc, optionally in the form of zinc oxide; about 2 mg copper, optionally in the form of copper oxide; about 10 mg R′R′ lutein; about 2 mg R′R′ zeaxanthin; and about 100 mg coenzyme Q10, optionally in the form of ubiquinol.

Also disclosed herein are compositions comprising about 500 mg vitamin C, optionally in the form of ascorbic acid; about 400 mg vitamin E, optionally in the form of dl-alpha tocopheryl acetate; about 80 mg zinc, optionally in the form of zinc oxide; about 2 mg copper, optionally in the form of copper oxide; about 10 mg R′R′ lutein; about 2 mg R′R′ zeaxanthin; and about 100 mg coenzyme Q10, optionally in the form of ubiquinone.

Also disclosed herein are compositions comprising about 500 mg vitamin C, optionally in the form of ascorbic acid; about 400 mg vitamin E, optionally in the form of dl-alpha tocopheryl acetate; about 25 mg zinc, optionally in the form of zinc oxide; about 2 mg copper, optionally in the form of copper oxide; about 10 mg R′R′ lutein; about 2 mg R′R′ zeaxanthin; and about 100 mg coenzyme Q10, optionally in the form of ubiquinol.

Also disclosed herein are compositions comprising about 500 mg vitamin C, optionally in the form of ascorbic acid; about 400 mg vitamin E, optionally in the form of dl-alpha tocopheryl acetate; about 25 mg zinc, optionally in the form of zinc oxide; about 2 mg copper, optionally in the form of copper oxide; about 10 mg R′R′ lutein; about 2 mg R′R′ zeaxanthin; and about 100 mg coenzyme Q10, optionally in the form of ubiquinone.

Also disclosed herein are compositions comprising about 500 mg vitamin C, optionally in the form of ascorbic acid; about 180 mg vitamin E, optionally in the form of dl-alpha tocopheryl acetate; about 80 mg zinc, optionally in the form of zinc oxide; about 2 mg copper, optionally in the form of copper oxide; about 10 mg R′R′ lutein; about 2 mg R′R′ zeaxanthin; and about 100 mg coenzyme Q10, optionally in the form of ubiquinol.

Also disclosed herein are compositions comprising about 500 mg vitamin C, optionally in the form of ascorbic acid; about 180 mg vitamin E, optionally in the form of dl-alpha tocopheryl acetate; about 80 mg zinc, optionally in the form of zinc oxide; about 2 mg copper, optionally in the form of copper oxide; about 10 mg R′R′ lutein; about 2 mg R′R′ zeaxanthin; and about 100 mg coenzyme Q10, optionally in the form of ubiquinone.

Also disclosed herein are compositions comprising about 500 mg vitamin C, optionally in the form of ascorbic acid; about 180 mg vitamin E, optionally in the form of dl-alpha tocopheryl acetate; about 25 mg zinc, optionally in the form of zinc oxide; about 2 mg copper, optionally in the form of copper oxide; about 10 mg R′R′ lutein; about 2 mg R′R′ zeaxanthin; and about 100 mg coenzyme Q10, optionally in the form of ubiquinol.

Also disclosed herein are compositions comprising about 500 mg vitamin C, optionally in the form of ascorbic acid; about 180 mg vitamin E, optionally in the form of dl-alpha tocopheryl acetate; about 25 mg zinc, optionally in the form of zinc oxide; about 2 mg copper, optionally in the form of copper oxide; about 10 mg R′R′ lutein; about 2 mg R′S′ zeaxanthin; and about 100 mg coenzyme Q10, optionally in the form of ubiquinone.

Also disclosed herein are compositions comprising about 500 mg vitamin C, optionally in the form of ascorbic acid; about 400 mg vitamin E, optionally in the form of dl-alpha tocopheryl acetate; about 80 mg zinc, optionally in the form of zinc oxide; about 2 mg copper, optionally in the form of copper oxide; about 10 mg R′R′ lutein; about 2 mg R′S′ zeaxanthin; and about 100 mg coenzyme Q10, optionally in the form of ubiquinol.

Also disclosed herein are compositions comprising about 500 mg vitamin C, optionally in the form of ascorbic acid; about 400 mg vitamin E, optionally in the form of dl-alpha tocopheryl acetate; about 80 mg zinc, optionally in the form of zinc oxide; about 2 mg copper, optionally in the form of copper oxide; about 10 mg R′R′ lutein; about 2 mg R′S′ zeaxanthin; and about 100 mg coenzyme Q10, optionally in the form of ubiquinone.

Also disclosed herein are compositions comprising about 500 mg vitamin C, optionally in the form of ascorbic acid; about 400 mg vitamin E, optionally in the form of dl-alpha tocopheryl acetate; about 25 mg zinc, optionally in the form of zinc oxide; about 2 mg copper, optionally in the form of copper oxide; about 10 mg R′R′ lutein; about 2 mg R′S′ zeaxanthin; and about 100 mg coenzyme Q10, optionally in the form of ubiquinol.

Also disclosed herein are compositions comprising about 500 mg vitamin C, optionally in the form of ascorbic acid; about 400 mg vitamin E, optionally in the form of dl-alpha tocopheryl acetate; about 25 mg zinc, optionally in the form of zinc oxide; about 2 mg copper, optionally in the form of copper oxide; about 10 mg R′R′ lutein; about 2 mg R′S′ zeaxanthin; and about 100 mg coenzyme Q10, optionally in the form of ubiquinone.

Also disclosed herein are compositions comprising about 500 mg vitamin C, optionally in the form of ascorbic acid; about 180 mg vitamin E, optionally in the form of dl-alpha tocopheryl acetate; about 80 mg zinc, optionally in the form of zinc oxide; about 2 mg copper, optionally in the form of copper oxide; about 10 mg R′R′ lutein; about 2 mg R′S′ zeaxanthin; and about 100 mg coenzyme Q10, optionally in the form of ubiquinol.

Also disclosed herein are compositions comprising about 500 mg vitamin C, optionally in the form of ascorbic acid; about 180 mg vitamin E, optionally in the form of dl-alpha tocopheryl acetate; about 80 mg zinc, optionally in the form of zinc oxide; about 2 mg copper, optionally in the form of copper oxide; about 10 mg R′R′ lutein; about 2 mg R′S′ zeaxanthin; and about 100 mg coenzyme Q10, optionally in the form of ubiquinone.

Also disclosed herein are compositions comprising about 500 mg vitamin C, optionally in the form of ascorbic acid; about 180 mg vitamin E, optionally in the form of dl-alpha tocopheryl acetate; about 25 mg zinc, optionally in the form of zinc oxide; about 2 mg copper, optionally in the form of copper oxide; about 10 mg R′R′ lutein; about 2 mg R′S′ zeaxanthin; and about 100 mg coenzyme Q10, optionally in the form of ubiquinol.

Also disclosed herein are compositions comprising about 500 mg vitamin C, optionally in the form of ascorbic acid; about 180 mg vitamin E, optionally in the form of dl-alpha tocopheryl acetate; about 25 mg zinc, optionally in the form of zinc oxide; about 2 mg copper, optionally in the form of copper oxide; about 10 mg R′R′ lutein; about 2 mg R′S′ zeaxanthin; and about 100 mg coenzyme Q10, optionally in the form of ubiquinone.

Age-related macular degeneration (AMD) is a leading cause of severe visual acuity loss in the United States and Western Europe in persons aged 55 years or older. An estimated 1.75 million individuals in the United States have advanced AMD, which accounts for most cases of severe vision loss. An additional 7.3 million persons have early AMD, which is usually associated with little or no vision loss but increases the risk of developing AMD. It is associated with a collection of clinically recognizable ocular findings that can lead to blindness. These findings include drusen, retinal pigment epithelial (RPE) disturbance, including pigment clumping and/or dropout, RPE detachment, geographic atrophy, subretinal neovascularization and disciform scar. Not all these manifestations are needed for AMD to be considered present.

Disclosed herein are compositions for eye and/or heart health, for example, for preventing or treating macular degeneration and/or heart disease. In some embodiments, the compositions disclosed herein are nutritional or dietary supplement compositions. In some embodiments, the compositions are pharmaceutical compositions. In some embodiments, the disclosure relates to antioxidant nutritional supplements comprising coenzyme Q10. In some embodiments, the compositions disclosed herein may strengthen and/or promote retinal health, for example through stabilization and/or treatment of visual acuity loss in people with particular ocular diseases, disorders, and/or conditions. In some embodiments, the compositions disclosed herein may improve and/or promote heart health, for example by treating and/or preventing the onset of heart failure and/or other cardiac risks. In some embodiments, administration of the compositions may decrease visual acuity loss, for example by reducing the risk of developing late stage or advanced age-related macular degeneration in patients. In some embodiments, a composition of the present disclosure simultaneously promotes eye health and heart health.

Also disclosed herein are methods for treatment and/or prevention of at least one disease, disorder, and/or condition associated with loss of visual acuity, the method comprising administering to a subject in need thereof a composition as disclosed herein. In some embodiments, the at least one disease, disorder, and/or condition associated with a loss of visual acuity is chosen from macular degeneration, age-related macular degeneration (AMD), atrophy of retinal pigmented epithelium (RPE), atrophy of at least one photoreceptor, drusen, drusenoid pigment epithelial detachment (PED), diabetic retinopathy, cataracts, retinitis pigmentosa, glaucoma, choroidal neovascularization, retinal degeneration, and oxygen-induced retinopathy. In some embodiments, the methods for treatment and/or prevention of at least one disease, disorder, and/or condition associated with loss of visual acuity are further treatment and/or prevention of at least one disease, disorder, and/or condition associated with heart health.

Also disclosed herein are methods of regressing drusen and/or drusenoid pigment epithelial detachment (PED), methods for treating and/or preventing atrophy of retinal pigmented epithelium (RPE) and/or at least one photoreceptor, methods for treating and/or preventing vision loss and/or improving acuity, and/or methods for treating and/or preventing at least one heart disease, disorder, or condition. The methods comprise administering to a subject in need thereof a composition as disclosed herein.

According to the present disclosure, there is provided compositions and methods to improve eye health and/or slowdown progression of macular degeneration. In some embodiments, there is also provided compositions and methods to improve heart health and/or slow the onset or progression of heart disease. In some embodiments, the compositions and methods of the present disclosure improve both eye and heart health.

Additionally, combining coenzyme Q10 of the present disclosure with other ingredients, such as AREDS ingredients, like zinc, copper, lutein, zeaxanthin, vitamin C and vitamin E, may also offer protection against macular degeneration and/or heart failure. These ingredients are potent antioxidants that help protect cells, such as retinal pigment endothelial cells (RPE) that make up the retina and are responsible for visual function and can lower blood pressure and treat heart failure and other heart conditions. Thus, coenzyme Q10 in conjunction with AREDS vitamins will help ensure that RPE cells are not further damaged, or less stressed, when subjects are given supplements containing the combination. This combination of coenzyme Q10 and AREDS nutrients should promote improved ocular health and slower progression of AMD and may also be used to treat heart failure and/or other heart conditions, possibly preventing at least one cardiac disease, disorder, and/or condition.

Definitions of certain terms as used in this application are provided below. Unless defined otherwise, all technical and scientific terms used herein have the normal and common meaning that would be commonly understood by one of ordinary skill in the art to which this disclosure belongs.

As used herein, “a,” “an,” and “the” refer to one or more (i.e., to at least one) of the grammatical object of the article.

As used herein, administration of a “daily” amount of a recited element or composition refers to the total amount that is administered in one day but does not limit the frequency of administration per day. The daily amount administered to a patient can be administered once or multiple times in a day, such as twice daily or three times daily (wherein each of multiple administrations comprises administering some amount of a recited element or composition that is less than the “daily” amount, given that the “daily” amount refers to the total amount administered in one day). Each administration of a recited element or composition can consist of administering the recited element or composition in the form of a single dosage (e.g., such as a single tablet or a single capsule) or in the form of multiple dosages (e.g., such as multiple (i.e., two or more) tablets and/or capsules).

As used herein, the terms “treat” and “treatment” include medical management of a disease, disorder, and/or condition of a subject as would be understood by a person of ordinary skill in the art (see, e.g., Stedman's Medical Dictionary). In general, an appropriate dose and treatment regimen provide at least one of the compositions of the present disclosure sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or lessen an undesired physiological change or disorder, or to prevent or slow or lessen the expansion or severity of such disorder. As discussed herein, beneficial or desired clinical results from treating a subject include, but are not limited to, abatement, lessening, or alleviation of symptoms that result from or are associated with the disease, condition, and/or disorder to be treated; decreased occurrence of symptoms; improved quality of life; longer symptom-free status (i.e., decreasing the likelihood or the propensity that a subject will present symptoms on the basis of which a diagnosis of a disease is made); diminishment of extent of disease, disorder, and/or condition; stabilized (i.e., not worsening) state of disease, disorder, and/or condition; delay or slowing of progression of a disease, disorder, and/or condition; amelioration or palliation of the state of a disease, disorder, and/or condition; and remission (whether partial or total), whether detectable or undetectable; and/or overall survival.

As used herein, “prevention” of or “preventing” a disorder or condition refers to reduction of or reducing the occurrence of the disorder or condition in a treated sample relative to an untreated control sample, and includes delaying onset, progression, or reduction of severity of one or more symptoms of the disorder or condition relative to the untreated control sample.

As used herein, the term “pharmaceutically acceptable salt” refers to a salt form of a compound wherein the salt is nontoxic and include such salts derived from suitable inorganic and organic acids and bases. Pharmaceutically acceptable salts are well known in the art. For example, S. M. Berge, et al. describes pharmaceutically acceptable salts in detail in J. Pharmaceutical Sciences, 1977, 66, 1-19. Non-limiting examples of suitable pharmaceutically acceptable acid addition salts include chlorides, bromides, sulfates, nitrates, phosphates, sulfonates, methane sulfonates, formates, tartrates, maleates, succinates, malonates, citrates, benzoates, salicylates, and ascorbates. Non-limiting examples of suitable pharmaceutically acceptable base addition salts include sodium, potassium, lithium, ammonium (substituted and unsubstituted), calcium, magnesium, iron, zinc, copper, manganese, and aluminum salts. Non-limiting examples of pharmaceutically acceptable salts include pharmaceutically acceptable salts derived from appropriate bases and include alkali metal, alkaline earth metal, ammonium, and N⁺(C₁₋₄alkyl)₄ salts. This disclosure also envisions the quaternization of any basic nitrogen-containing groups of the compounds disclosed herein. Pharmaceutically acceptable salts may, for example, be obtained using standard procedures well known in the field of pharmaceuticals. One of ordinary skill in the art will recognize that the stability and other properties of different pharmaceutically acceptable salts of the recited components herein may differ and will consider these differences when selecting suitable pharmaceutically acceptable salt(s).

The terms “patient,” “subject,” “individual,” and the like, as used herein, are interchangeable and refer to any animal, which may be a human or a non-human animal.

As used herein, “age-related macular degeneration” or “AMD” includes all forms of macular degeneration, including “wet” (exudative) and “dry” (atrophic) forms.

The term “prodrug” includes compounds that may be converted (e.g., under physiological conditions or by solvolysis) to a biologically active compound. Thus, the term “prodrug” includes metabolic precursors of compounds that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release active compound(s) as described herein in vivo when such prodrug is administered to a subject. Non-limiting examples of prodrugs include ester and amide derivatives of hydroxy, carboxy, mercapto and amino functional groups in the compounds described herein.

The term “nutritional composition,” as used herein, includes a food product intended for human consumption.

As will be understood by one of ordinary skill in the art, when disclosed herein, each range includes all possible subranges as well as individual numerical values within that range. For example, a range of “1.0 to 5.0” includes and would be understood to specifically disclose subranges such as “1.0 to 3.0,” “1.5 to 3.7,” “2.1 to 4.3, etc., as well as all individual numbers within the disclosed range, for example, 1.0, 1.1, 1.2, 1.3, etc.

Disclosed herein are compositions comprising a vitamin complex comprising:
vitamin C, a prodrug thereof, and/or a pharmaceutically acceptable salt of any of the foregoing;
vitamin E, a prodrug thereof, and/or a pharmaceutically acceptable salt of any of the foregoing;
zinc, a prodrug thereof, and/or a pharmaceutically acceptable salt of any of the foregoing;
copper, a prodrug thereof, and/or a pharmaceutically acceptable salt of any of the foregoing;
lutein, a prodrug thereof, and/or a pharmaceutically acceptable salt of any of the foregoing;
zeaxanthin, a prodrug thereof, and/or a pharmaceutically acceptable salt of any of the foregoing; and
coenzyme Q10, a prodrug thereof, and/or a pharmaceutically acceptable salt of any of the foregoing.

In some embodiments, the vitamin C, a prodrug thereof, and/or a pharmaceutically acceptable salt of any of the foregoing comprises vitamin C, optionally in the form of ascorbic acid. In some embodiments, the vitamin E, a prodrug thereof, and/or a pharmaceutically acceptable salt of any of the foregoing comprises dl-alpha tocopheryl acetate and/or alpha-tocopherol. In some embodiments, the zinc, a prodrug thereof, and/or a pharmaceutically acceptable salt of any of the foregoing comprises zinc oxide and/or zinc gluconate. In some embodiments, the copper, a prodrug thereof, and/or a pharmaceutically acceptable salt of any of the foregoing comprises copper oxide and/or copper gluconate. In some embodiments, the zeaxanthin, a prodrug thereof, and/or a pharmaceutically acceptable salt of any of the foregoing comprises zeaxanthin (R′R′). In some embodiments, the coenzyme Q10, a prodrug thereof, and/or a pharmaceutically acceptable salts of any of the foregoing comprises coenzyme Q10, optionally in the form of ubiquinol.

In some embodiments, the composition comprises vitamin C, optionally in the form of ascorbic acid, vitamin E, optionally in the form of dl-alpha tocopheryl acetate, zinc, optionally in the form of zinc oxide, thiamin, copper, optionally in the form of copper oxide, R′R′ lutein, R′R′ zeaxanthin, and coenzyme Q10, optionally in the form of ubiquinol.

In some embodiments, the vitamin C, a prodrug thereof, and/or a pharmaceutically acceptable salt of any of the foregoing comprises ascorbic acid. In some embodiments, the vitamin C, a prodrug thereof, and/or a pharmaceutically acceptable salt of any of the foregoing is ascorbic acid. The U.S. recommended dietary allowance (RDA) for vitamin C in the form of ascorbic acid is 60 mg. In some embodiments, the composition comprises 60 mg to 850 mg of vitamin C or an equivalent amount of a prodrug, a pharmaceutically acceptable salt, and/or a pharmaceutically acceptable salt of a prodrug of vitamin C. In some embodiments, the composition comprises 100.0 mg to 800.0 mg, such as 150.0 mg to 800.0 mg, 250.0 mg to 800.0 mg, 250.0 mg to 750.0 mg, 500.0 mg to 775.0 mg, 600.0 mg to 750.0 mg, 500.0 mg, or 750.0 mg, of ascorbic acid or an equivalent amount of a prodrug, a pharmaceutically acceptable salt, and/or a pharmaceutically acceptable salt of a prodrug of vitamin C. In some embodiments, the composition comprises 500.0 mg of vitamin C, optionally in the form of ascorbic acid. In some embodiments, the composition comprises about 60 mg to about 850 mg of vitamin C or an equivalent amount of a prodrug, a pharmaceutically acceptable salt, and/or a pharmaceutically acceptable salt of a prodrug of vitamin C. In some embodiments, the composition comprises about 100 mg to about 800 mg, such as about 150 mg to about 800 mg, about 250 mg to about 800 mg, about 250 mg to about 750 mg, about 500 mg to about 775 mg, about 600 mg to about 750 mg, about 500 mg, or about 750 mg, of ascorbic acid or an equivalent amount of a prodrug, a pharmaceutically acceptable salt, and/or a pharmaceutically acceptable salt of a prodrug of vitamin C. In some embodiments, the composition comprises about 500 mg of vitamin C, optionally in the form of ascorbic acid.

In some embodiments, the vitamin E, a prodrug thereof, and/or a pharmaceutically acceptable salt of any of the foregoing comprises alpha-tocopherol. In some embodiments, the vitamin E, a prodrug thereof, and/or a pharmaceutically acceptable salt of any of the foregoing is dl-alpha tocopheryl acetate. The RDA of vitamin E is 15.0 mg. No adverse effects of vitamin E have been observed at levels as high as 800.0 mg. In some embodiments, the composition comprises 15.0 mg to 800 mg vitamin E or an equivalent amount of a prodrug, a pharmaceutically acceptable salt, and/or a pharmaceutically acceptable salt of a prodrug of vitamin E. In some embodiments, the composition comprises 15.0 mg to 800.0 mg vitamin E, such as 50.0 mg to 750.0 mg, 90.0 mg to 600.0 mg, 180.0 mg to 500.0 mg, 250.0 mg to 400.0 mg, 300.0 mg to 350.0 mg vitamin E or an equivalent amount of a prodrug, a pharmaceutically acceptable salt, and/or a pharmaceutically acceptable salt of a prodrug of vitamin E. In some embodiments, the composition comprises 180 mg vitamin E. In some embodiments, the composition comprises about 15 mg to about 800 mg vitamin E or an equivalent amount of a prodrug, a pharmaceutically acceptable salt, and/or a pharmaceutically acceptable salt of a prodrug of vitamin E. In some embodiments, the composition comprises about 15 mg to about 800 mg vitamin E, such as about 50 mg to about 750 mg, about 90 mg to about 600 mg, about 180 mg to about 500 mg, about 250 mg to about 400 mg, about 300 mg to about 350 mg vitamin E or an equivalent amount of a prodrug, a pharmaceutically acceptable salt, and/or a pharmaceutically acceptable salt of a prodrug of vitamin E. In some embodiments, the composition comprises about 180 mg vitamin E, optionally in the form of dl-alpha tocopheryl acetate.

In some embodiments, the zinc, a prodrug thereof, and/or a pharmaceutically acceptable form of any of the foregoing comprises zinc oxide. In some embodiments, the zinc, a prodrug thereof, and/or a pharmaceutically acceptable form of any of the foregoing is zinc oxide. The RDA for zinc is about 15 mg. In some embodiments, the composition comprises 15 mg to 100 mg of zinc or an equivalent amount of a prodrug, a pharmaceutically acceptable salt, and/or a pharmaceutically acceptable form of a prodrug of zinc. In some embodiments, the composition comprises 15.0 mg to 100.0 mg, such as 40.0 mg to 95.0 mg, 50.0 mg to 90.0 mg, 60.0 mg to 85.0 mg, 70.0 mg to 80.0 mg, or 80.0 mg, zinc or an equivalent amount of a prodrug, a pharmaceutically acceptable form, and/or a pharmaceutically acceptable salt of a prodrug of zinc. In some embodiments, the composition comprises 80.0 mg of zinc, optionally in the form of zinc oxide. In some embodiments, the composition comprises 25.0 mg zinc, optionally in the form of zinc oxide. In some embodiments, the composition comprises about 15 mg to about 100 mg of zinc or an equivalent amount of a prodrug, a pharmaceutically acceptable salt, and/or a pharmaceutically acceptable form of a prodrug of zinc. In some embodiments, the composition comprises about 15 mg to about 100 mg, such as about 40 mg to about 95 mg, about 50 mg to about 90 mg, about 60 mg to about 85.0 mg, about 70 mg to about 80 mg, or about 80 mg, zinc or an equivalent amount of a prodrug, a pharmaceutically acceptable form, and/or a pharmaceutically acceptable salt of a prodrug of zinc. In some embodiments, the composition comprises about 80 mg of zinc, optionally in the form of zinc oxide. In some embodiments, the composition comprises about 25 mg zinc, optionally in the form of zinc oxide.

In some embodiments, the copper, a prodrug thereof, and/or a pharmaceutically acceptable form of any of the foregoing comprises copper oxide. In some embodiments, the copper, a prodrug thereof, and/or a pharmaceutically acceptable form of any of the foregoing is copper oxide. The RDA for copper is 2.0 mg. In some embodiments, the composition comprises 1.5 mg to 2.5 mg of copper or an equivalent amount of a prodrug, a pharmaceutically acceptable form, and/or a pharmaceutically acceptable salt of a prodrug of copper. In some embodiments, the composition comprises 1.5 mg to 2.0 mg, such as 2.0 mg, of copper or an equivalent amount of a prodrug, a pharmaceutically acceptable form, and/or a pharmaceutically acceptable salt of a prodrug of copper. In some embodiments, the composition comprises 2.0 mg of copper, optionally in the form of copper oxide. In some embodiments, the composition comprises about 1.5 mg to about 2.5 mg of copper or an equivalent amount of a prodrug, a pharmaceutically acceptable form, and/or a pharmaceutically acceptable salt of a prodrug of copper. In some embodiments, the composition comprises about 1.5 mg to about 2.0 mg, such as about 2.0 mg of copper or an equivalent amount of a prodrug, a pharmaceutically acceptable form, and/or a pharmaceutically acceptable salt of a prodrug of copper. In some embodiments, the composition comprises about 2.0 mg of copper, optionally in the form of copper oxide.

In some embodiments, the composition further comprises lutein, a prodrug thereof, and/or a pharmaceutically acceptable salt of lutein. In some embodiments, the lutein, a prodrug thereof, and/or a pharmaceutically acceptable salt of lutein is R′R′-lutein. Lutein is a carotenoid. In some embodiments, the composition comprises 5.0 mg to 50.0 mg, such as 5.0 mg to 15.0 mg of lutein or an equivalent amount of a prodrug, a pharmaceutically acceptable salt, and/or a pharmaceutically acceptable salt of a prodrug of lutein. In some embodiments, the composition comprises 5.0 mg to 50.0 mg, such as 6.0 mg to 40.0 mg, 7.0 mg to 30.0 mg, 8.0 mg to 20.0 mg, 9.0 mg to 11.0 mg, 20.0 mg to 30.0 mg, 30.0 mg to 40.0 mg, 40.0 mg to 50.0 mg, 5.0 mg to 15.0 mg, 6.0 mg to 14.0 mg, 7.0 mg to 13.0 mg, 8.0 mg to 12.0 mg, 9.0 mg to 11.0 mg, 10.0 mg, 20.0 mg, 30.0 mg, 40.0 mg, or 50.0 mg, of lutein or an equivalent amount of a prodrug, a pharmaceutically acceptable salt, and/or a pharmaceutically acceptable salt of a prodrug of lutein. In some embodiments, the composition comprises 10.0 mg of lutein. In some embodiments, the composition comprises about 5 mg to about 50 mg, such as about 5 mg to about 15 mg of lutein or an equivalent amount of a prodrug, a pharmaceutically acceptable salt, and/or a pharmaceutically acceptable salt of a prodrug of lutein. In some embodiments, the composition comprises about 5 mg to about 50 mg, such as about 5 mg to about 15 mg, about 6 mg to about 40 mg, about 6 mg to about 14 mg, about 7 mg to about 30 mg, about 7 mg to about 13 mg, about 8 mg to about 20 mg, about 8 mg to about 12 mg, about 9 mg to about 11 mg, about 20 mg to about 30 mg, about 30 mg to about 40 mg, about 40 mg to about 50 mg, about 10 mg, about 20 mg, about 30 mg, about 40 mg, or about 50 mg, of lutein or an equivalent amount of a prodrug, a pharmaceutically acceptable salt, and/or a pharmaceutically acceptable salt of a prodrug of lutein. In some embodiments, the composition comprises about 10 mg of lutein.

In some embodiments, the composition further comprises zeaxanthin, a prodrug thereof, and/or a pharmaceutically acceptable salt of zeaxanthin. In some embodiments, the zeaxanthin, a prodrug thereof, and/or a pharmaceutically acceptable salt of zeaxanthin is R′,R′-zeaxanthin. In some embodiments, the zeaxanthin, a prodrug thereof, and/or a pharmaceutically acceptable salt of zeaxanthin is R′,S′-zeaxanthin. Zeaxanthin is a carotenoid. In some embodiments, the composition comprises 0.01 mg to 40.0 mg, such as 1.5 mg to 2.5 mg of zeaxanthin or an equivalent amount of a prodrug, a pharmaceutically acceptable salt, and/or a pharmaceutically acceptable salt of a prodrug of zeaxanthin. In some embodiments, the composition comprises 0.04 mg to 40.0 mg, such as 1.0 mg to 30.0 mg, 3.0 mg to 25.0 mg, 5.0 mg to 20.0 mg, 10.0 mg to 20.0 mg, 1.0 mg to 15.0 mg, 3.0 mg to 12.0 mg, 5.0 mg to 20.0 mg, 7.0 mg to 19.0 mg, 8.0 mg to 15.0 mg, 1.5 mg to 2.5 mg, 1.5 mg to 2.0 mg, 2.0 mg to 2.5 mg, or 2.0 mg, of zeaxanthin or an equivalent amount of a prodrug, a pharmaceutically acceptable salt, and/or a pharmaceutically acceptable salt of a prodrug of zeaxanthin. In some embodiments, the composition comprises 2.0 mg of zeaxanthin. In some embodiments, the composition comprises about 1 mg to about 20 mg, such as about 1.5 mg to about 2.5 mg of zeaxanthin or an equivalent amount of a prodrug, a pharmaceutically acceptable salt, and/or a pharmaceutically acceptable salt of a prodrug of zeaxanthin. In some embodiments, the composition comprises about 1 mg to about 20 mg, such as about 1 mg to about 3 mg, about 3 mg to about 5 mg, about 1.5 mg to about 2.5 mg, such as about 1.5 mg to about 2 mg, about 2 mg to about 2.5 mg, about 5 mg to about 7 mg, about 7 mg to about 9 mg, about 9 mg to about 11 mg, about 11 mg to about 13 mg, about 13 mg to about 15 mg, about 15 mg to about 17 mg about 17 mg to about 19 mg, about 2 mg, about 3 mg, about 4 mg, about 5, mg, about 10 mg, or about 20 mg, of zeaxanthin or an equivalent amount of a prodrug, a pharmaceutically acceptable salt, and/or a pharmaceutically acceptable salt of a prodrug of zeaxanthin. In some embodiments, the composition comprises about 2 mg of zeaxanthin.

In some embodiments, the composition further comprises coenzyme Q10, a prodrug thereof, and/or a pharmaceutically acceptable salt of coenzyme Q10. In some embodiments, the coenzyme Q10, a prodrug thereof, and/or a pharmaceutically acceptable salt of coenzyme Q10 is ubiquinone. In some embodiments, the coenzyme Q10, a prodrug thereof, and/or a pharmaceutically acceptable salt of coenzyme Q10 is ubiquinol. In some embodiments, the composition comprises 50 mg to 500 mg, such as 55 mg to 475 mg of coenzyme Q10 or an equivalent amount of a prodrug, a pharmaceutically acceptable salt, and/or a pharmaceutically acceptable salt of a prodrug of coenzyme Q10. In some embodiments, the composition comprises 60 mg to 450 mg, such as 65 mg to 425 mg, 70 mg to 400 mg, 75 mg to 375 mg, 80 mg to 350 mg, 85 mg to 325 mg, 90 mg to 300 mg, 95 mg to 275 mg, 100 mg to 250 mg, 100 mg to 200 mg, 100 mg to 175 mg, 100 mg to 150 mg, 100 mg to 125 mg, or 100 mg, of coenzyme Q10 or an equivalent amount of a prodrug, a pharmaceutically acceptable salt, and/or a pharmaceutically acceptable salt of a prodrug of coenzyme Q10. In some embodiments, the composition comprises 100 mg of coenzyme Q10. In some embodiments, the composition comprises about 50 mg to about 500 mg, such as about 55 mg to about 475 mg of coenzyme Q10 or an equivalent amount of a prodrug, a pharmaceutically acceptable salt, and/or a pharmaceutically acceptable salt of a prodrug of coenzyme Q10. In some embodiments, the composition comprises about 60 mg to about 450 mg, such as about 65 mg to about 400 mg, about 70 mg to about 375 mg, about 75 mg to about 350 mg, about 80 mg to about 325 mg, about 85 mg to about 300 mg, about 90 mg to about 275 mg, about 95 mg to about 250 mg, about 100 mg to about 225 mg, about 100 mg to about 200 mg, about 100 mg to about 175 mg, about 100 mg to about 150 mg, about 100 mg to about 125 mg, or about 100 mg, of coenzyme Q10 or an equivalent amount of a prodrug, a pharmaceutically acceptable salt, and/or a pharmaceutically acceptable salt of a prodrug of coenzyme Q10. In some embodiments, the composition comprises about 100 mg of coenzyme Q10.

In some embodiments, the composition comprises:
350.0 mg to 800.0 mg of vitamin C or an equivalent amount of a prodrug, a pharmaceutically acceptable salt, and/or a pharmaceutically acceptable salt of a prodrug of vitamin C;
50.0 mg to 550.0 mg of vitamin E or an equivalent amount of a prodrug, a pharmaceutically acceptable salt, and/or a pharmaceutically acceptable salt of a prodrug of vitamin E;
20.0 mg to 90.0 mg of zinc or an equivalent amount of a prodrug, a pharmaceutically acceptable form, and/or a pharmaceutically acceptable salt of a prodrug of zinc;
1.5 mg to 2.5 mg of copper or an equivalent amount of a prodrug, a pharmaceutically acceptable form, and/or a pharmaceutically acceptable salt of a prodrug of copper;
5.0 mg to 50.0 mg of lutein or an equivalent amount of a prodrug, a pharmaceutically acceptable salt, and/or a pharmaceutically acceptable salt of a prodrug of lutein;
1.0 mg to 20.0 mg of zeaxanthin or an equivalent amount of a prodrug, a pharmaceutically acceptable salt, and/or a pharmaceutically acceptable salt of a prodrug of zeaxanthin; and
50 mg to 500 mg of coenzyme Q10 or an equivalent amount of a prodrug, a pharmaceutically acceptable salt, and/or a pharmaceutically acceptable salt of a prodrug of coenzyme Q10.

In some embodiments, the composition comprises: 500.0 mg vitamin C, optionally in the form of ascorbic acid; 400.0 mg vitamin E, optionally in the form of dl-alpha tocopheryl acetate; 80.0 mg zinc, optionally in the form of zinc oxide; 2.0 mg copper, optionally in the form of copper oxide; 10.0 mg R′,R′ lutein; 2.0 mg R′,R′ zeaxanthin; and/or 100 mg coenzyme Q10, optionally in the form of ubiquinol.

In some embodiments, the composition comprises: 500.0 mg vitamin C, optionally in the form of ascorbic acid; 400.0 mg vitamin E, optionally in the form of dl-alpha tocopheryl acetate; 80.0 mg zinc, optionally in the form of zinc oxide; 2.0 mg copper, optionally in the form of copper oxide; 10.0 mg R′,R′ lutein; 2.0 mg R′,R′ zeaxanthin; and/or 100 mg coenzyme Q10, optionally in the form of ubiquinone.

In some embodiments, the composition comprises: 500.0 mg vitamin C, optionally in the form of ascorbic acid; 400.0 mg vitamin E, optionally in the form of dl-alpha tocopheryl acetate; 25.0 mg zinc, optionally in the form of zinc oxide; 2.0 mg copper, optionally in the form of copper oxide; 10.0 mg R′,R′ lutein; 2.0 mg R′,R′ zeaxanthin; and/or coenzyme Q10, optionally in the form of ubiquinol.

In some embodiments, the composition comprises: 500.0 mg vitamin C, optionally in the form of ascorbic acid; 400.0 mg vitamin E, optionally in the form of dl-alpha tocopheryl acetate; 25.0 mg zinc, optionally in the form of zinc oxide; 2.0 mg copper, optionally in the form of copper oxide; 10.0 mg R′,R′ lutein; 2.0 mg R′,R′ zeaxanthin; and/or coenzyme Q10, optionally in the form of ubiquinone.

In some embodiments, the composition comprises: 500.0 mg vitamin C, optionally in the form of ascorbic acid; 180.0 mg vitamin E, optionally in the form of dl-alpha tocopheryl acetate; 80.0 mg zinc, optionally in the form of zinc oxide; 2.0 mg copper, optionally in the form of copper oxide; 10.0 mg R′,R′ lutein, 2.0 mg R′,R′ zeaxanthin; and 100 mg coenzyme Q10, optionally in the form of ubiquinol.

In some embodiments, the composition comprises: 500.0 mg vitamin C, optionally in the form of ascorbic acid; 180.0 mg vitamin E, optionally in the form of dl-alpha tocopheryl acetate; 80.0 mg zinc, optionally in the form of zinc oxide; 2.0 mg copper, optionally in the form of copper oxide; 10.0 mg R′,R′ lutein, 2.0 mg R′,R′ zeaxanthin; and 100 mg coenzyme Q10, optionally in the form of ubiquinone.

In some embodiments, the composition comprises: 500.0 mg vitamin C, optionally in the form of ascorbic acid; 180.0 mg vitamin E, optionally in the form of dl-alpha tocopheryl acetate; 25.0 mg zinc, optionally in the form of zinc oxide; 2.0 mg copper, optionally in the form of copper oxide; 10.0 mg R′,R′ lutein; 2.0 mg R′,R′ zeaxanthin; and 100 mg coenzyme Q10, optionally in the form of ubiquinol.

In some embodiments, the composition comprises: 500.0 mg vitamin C, optionally in the form of ascorbic acid; 180.0 mg vitamin E, optionally in the form of dl-alpha tocopheryl acetate; 25.0 mg zinc, optionally in the form of zinc oxide; 2.0 mg copper, optionally in the form of copper oxide; 10.0 mg R′,R′ lutein; 2.0 mg R′,R′ zeaxanthin; and 100 mg coenzyme Q10, optionally in the form of ubiquinone

In some embodiments, the composition comprises
about 350 mg to about 800 mg of vitamin C or an equivalent amount of a prodrug, a pharmaceutically acceptable salt, and/or a pharmaceutically acceptable salt of a prodrug of vitamin C;
about 200 mg to about 550 mg of vitamin E or an equivalent amount of a prodrug, a pharmaceutically acceptable salt, and/or a pharmaceutically acceptable salt of a prodrug of vitamin E;
about 20 mg to about 90 mg of zinc or an equivalent amount of a prodrug, a pharmaceutically acceptable form, and/or a pharmaceutically acceptable salt of a prodrug of zinc;
about 1.5 mg to about 2.5 mg of copper or an equivalent amount of a prodrug, a pharmaceutically acceptable form, and/or a pharmaceutically acceptable salt of a prodrug of copper;
about 5 mg to about 50 mg of lutein or an equivalent amount of a prodrug, a pharmaceutically acceptable salt, and/or a pharmaceutically acceptable salt of a prodrug of lutein;
about 1 mg to about 20 mg of zeaxanthin or an equivalent amount of a prodrug, a pharmaceutically acceptable salt, and/or a pharmaceutically acceptable salt of a prodrug of zeaxanthin; and
about 20 to about 500 mg coenzyme Q10 or an equivalent amount of a prodrug, a pharmaceutically acceptable salt, and/or a pharmaceutically acceptable salt of a prodrug of coenzyme Q10.

In some embodiments, the composition comprises: about 500 mg vitamin C, optionally in the form of ascorbic acid; about 400 mg vitamin E, optionally in the form of dl-alpha tocopheryl acetate; about 80 mg zinc, optionally in the form of zinc oxide; about 2 mg copper, optionally in the form of copper oxide about 10 mg R′,R′ lutein; about 2 mg R′,R′ zeaxanthin; and about 100 mg coenzyme Q10, optionally in the form of ubiquinol.

In some embodiments, the composition comprises: about 500 mg vitamin C, optionally in the form of ascorbic acid; about 400 mg vitamin E, optionally in the form of dl-alpha tocopheryl acetate; about 80 mg zinc, optionally in the form of zinc oxide; about 2 mg copper, optionally in the form of copper oxide about 10 mg R′,R′ lutein; about 2 mg R′,R′ zeaxanthin; and about 100 mg coenzyme Q10, optionally in the form of ubiquinone.

In some embodiments, the composition comprises: about 500 mg vitamin C, optionally in the form of ascorbic acid; about 400 mg vitamin E, optionally in the form of dl-alpha tocopheryl acetate; about 25 mg zinc, optionally in the form of zinc oxide; about 2 mg copper, optionally in the form of copper oxide; about 10 mg R′,R′ lutein; about 2 mg R′,R′ zeaxanthin; and about 100 mg coenzyme Q10, optionally in the form of ubiquinol.

In some embodiments, the composition comprises: about 500 mg vitamin C, optionally in the form of ascorbic acid; about 400 mg vitamin E, optionally in the form of dl-alpha tocopheryl acetate; about 25 mg zinc, optionally in the form of zinc oxide; about 2 mg copper, optionally in the form of copper oxide; about 10 mg R′,R′ lutein; about 2 mg R′,R′ zeaxanthin; and about 100 mg coenzyme Q10, optionally in the form of ubiquinone.

In some embodiments, the composition comprises: about 500 mg vitamin C, optionally in the form of ascorbic acid; about 180 mg vitamin E, optionally in the form of dl-alpha tocopheryl acetate; about 80 mg zinc, optionally in the form of zinc oxide, about 2 mg copper, optionally in the form of copper oxide; about 10 mg R′,R′ lutein; about 2 mg R′,R′ zeaxanthin; and about 100 mg coenzyme Q10, optionally in the form of ubiquinol.

In some embodiments, the composition comprises: about 500 mg vitamin C, optionally in the form of ascorbic acid; about 180 mg vitamin E, optionally in the form of dl-alpha tocopheryl acetate; about 80 mg zinc, optionally in the form of zinc oxide, about 2 mg copper, optionally in the form of copper oxide; about 10 mg R′,R′ lutein; about 2 mg R′,R′ zeaxanthin; and about 100 mg coenzyme Q10, optionally in the form of ubiquinone.

In some embodiments, the composition comprises: about 500 mg vitamin C, optionally in the form of ascorbic acid; about 180 mg vitamin E, optionally in the form of dl-alpha tocopheryl acetate; about 25 mg zinc, optionally in the form of zinc oxide; about 2 mg copper, optionally in the form of copper oxide; about 10 mg R′,R′ lutein; about 2 mg R′,R′ zeaxanthin; and about 100 mg coenzyme Q10, optionally in the form of ubiquinol.

In some embodiments, the composition comprises: about 500 mg vitamin C, optionally in the form of ascorbic acid; about 180 mg vitamin E, optionally in the form of dl-alpha tocopheryl acetate; about 25 mg zinc, optionally in the form of zinc oxide; about 2 mg copper, optionally in the form of copper oxide; about 10 mg R′,R′ lutein; about 2 mg R′,R′ zeaxanthin; and about 100 mg coenzyme Q10, optionally in the form of ubiquinone.

In some embodiments, the compositions described herein may be a dietary or nutritional supplement or a pharmaceutical composition. In some embodiments, the compositions may be in the form of a food product, a component of a food product. The compositions of the present disclosure may advantageously be utilized in methods for promoting the health of an individual, such as eye and/or heart health.

In some embodiments, the composition is formulated in a form suitable for oral, intraperitoneal, intravenous, subcutaneous, sublingual, transcutaneous, and/or intramuscular administration. In some embodiments, the composition is formulated in a form suitable for oral administration. Non-limiting suitable solid oral formulations include tablets, capsules, cachets, lozenges, powders, pills, granules, and pellets. Non-limiting suitable liquid oral formulations include solutions, suspensions, dispersions, emulsions, and oils. In some embodiments, the composition is in liquid, semisolid or solid form. For example, the compositions may be administered as tablets, gel packs, capsules, gelatin capsules, flavored drinks, as a powder that can be reconstituted. In some embodiments, the composition is in the form of a tablet, capsule, soft gel, liquid, or powder. In some embodiments, the composition is a soft gelatin capsule and a hard gelatin capsule. In some embodiments, the composition is in the form of a chewable oral formulation, such as a chewable tablet.

In some embodiments, the composition is in the form of an immediate release formulation or a modified release formulation, such as a delayed release and/or an extended release formulation.

In some embodiments, the compositions may further comprise at least one pharmaceutically acceptable excipient. Non-limiting examples of suitable excipients include surfactants, humectants, plasticizers, binders, crystallization inhibitors, wetting agents, fillers, solubilizers, bioavailability enhancers, pH adjusting agents, and flavorants. The at least one pharmaceutically acceptable excipient, as used herein, also includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various excipients used in formulating oral compositions and known techniques for the preparation thereof. Except insofar as any conventional carrier is incompatible with the vitamins of this disclosure, such as by producing any undesirable biological effect or otherwise interacting in a deleterious manner with any other component(s) of the composition, its use is contemplated to be within the scope of this disclosure. Non-limiting examples of suitable pharmaceutically acceptable excipients include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

In some embodiments, the composition further comprises cellulose, gelatin, magnesium stearate, water, vegetable oil, glycerin, beeswax, and/or silica.

In some embodiments, the compositions further comprise or are co-administered with at least one additional active ingredient. In some embodiments, the at least one additional therapeutic agent is chosen from anti-inflammatory agents (e.g., anti-IL-6 agent, anti-IL-8 agents, aspirin, ibuprofen, and naproxen), anti-angiogenic agents (e.g., anti-VEGF agents, ranibizumab, bevacizumab, acadesine, and AMPK activators), anti-oxidative agents (e.g., vitamin C, vitamin E, vitamin A, glutathione, catalase, etc.), omega-3 fatty acids (e.g., alpha-linolenic acid (ALA), eicosapentaenoic acid (EPA), and docosahexaenoic acid (DHA)), and vitamins and minerals (e.g., vitamin C, vitamin E, vitamin A, lutein, zeaxanthin, zinc, and copper). In some embodiments, the composition further comprises lutein and zeaxanthin.

In some embodiments, the compositions disclosed herein further comprise B vitamins. In some embodiments, the compositions comprised herein further comprise omega-3 fatty acids. In some embodiments, the compositions disclosed herein further comprise very long chain polyunsaturated fatty acids.

Combination of the disclosed compositions and at least one additional active ingredient may advantageously produce one or more of the following effects: (1) additive and/or synergistic benefits; (2) reduction of the side effects and/or adverse effects associated with use of the prescription medicine in the absence of the compositions disclosed herein; and/or (3) the ability to lower the dosage of the prescription medicine in comparison to the amount of prescription medicine needed in the absence of the compositions disclosed herein.

The compositions disclosed herein may be prepared according to any known method for the manufacture of dietary supplements or pharmaceutical preparations. As will be appreciated by those of ordinary skill in the art, a number of methods are known. For information concerning materials, equipment and processes for preparing formulations and dosage forms, see “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et. al., (Media, Pa.: Williams and Wilkins, 1995) which provide information on carriers, materials, equipment and process for preparing formulations.

A method of manufacturing the compositions disclosed herein may comprise combining the recited vitamins as well as any desired excipients and mechanically mixing, such as for example, using a blender to form a blend. If necessary, the blend may be then tumbled until uniform. The blend may be then compressed using a tablet press to form tablets. Optionally a coating may be sprayed on the tablets and the tablets tumbled until dry. Alternatively, the blend may be placed in medium chain triglycerides to form a slurry for containment in a soft gel capsule, the blend may be placed in a gelatin capsule or the blend may be placed in other dosage forms known to those skilled in the art.

Methods for treating and/or preventing at least one disease, disorder, and/or condition associated with a loss of visual acuity including, for example, age-related macular degeneration (AMD), and/or heart health, such cardiac diseases are disclosed herein, the methods comprising administering to a subject a composition disclosed herein. In some embodiments, the composition disclosed herein is chosen from compositions comprising vitamin C, a prodrug thereof, and/or a pharmaceutically acceptable salt of any of the foregoing; vitamin E, a prodrug thereof, and/or a pharmaceutically acceptable salt of any of the foregoing; zinc, a prodrug thereof, and/or a pharmaceutically acceptable form of any of the foregoing; copper, a prodrug thereof, and/or a pharmaceutically acceptable form of any of the foregoing; lutein, a prodrug thereof, and/or a pharmaceutically acceptable salt of any of the foregoing; zeaxanthin, a prodrug thereof, and/or a pharmaceutically acceptable salt of any of the foregoing; and coenzyme Q10, a prodrug thereof, and/or a pharmaceutically acceptable salt of any of the foregoing.

The at least one disease, disorder, and/or condition associated with a loss of visual acuity includes age-related macular degeneration (AMD). In some embodiments, the method disclosed herein is effective to prevent, attenuate, or inhibit the progression of AMD. In some embodiments, AMD is wet AMD or dry AMD.

In some embodiments, the at least one disease, disorder, and/or condition associated with a loss of visual acuity is chosen from macular degeneration, age-related macular degeneration (AMD), atrophy of retinal pigmented epithelium (RPE), atrophy of at least one photoreceptor, drusen, drusenoid pigment epithelial detachment (PED), diabetic retinopathy, cataracts, retinitis pigmentosa, glaucoma, choroidal neovascularization, retinal degeneration, Stargardts disease, and oxygen-induced retinopathy.

In some embodiments, the at least one disease, disorder, and/or condition is associated with heart health. In some embodiments, the methods disclosed herein is effective to prevent, attenuate, or inhibit the progression of at least one eye disease, disorder, and/or condition and at least one heart disease, disorder, and/or condition.

In some embodiments of the methods disclosed herein, the compositions disclosed herein are administered to a subject in one, two, three, or four doses daily. In some embodiments, the compositions are each administered in the form of one, two, three, or four dosage units one, two, three, or four times daily, such as in the form of two tablets taken twice daily or in the form of one tablet taken twice daily.

Also disclosed herein are methods of regressing drusen and/or drusenoid pigment epithelial detachment (PED) comprising administering to a subject a composition disclosed herein. Methods for treating and/or preventing atrophy of retinal pigmented epithelium (RPE) and/or at least one photoreceptor are disclosed herein, wherein the methods comprise administering to a subject a composition disclosed herein. Methods for treating and/or preventing vision loss and/or improving acuity also disclosed herein. Methods for treating and/or preventing heart disease, disorder, and/or condition and/or improving heart health are also disclosed herein. In some embodiments, the methods disclosed herein are used for treating and/or preventing at least one eye disease, disorder, and/or condition and at least one heart disease, disorder, and/or condition.

In some embodiments, administration of a composition as described herein causes a complete disappearance (i.e., 100% regression) of the drusen. In some embodiments, administration of a composition as described herein prevents atrophy of the RPE and/or photoreceptors in a patient (i.e., geographic atrophy).

In some embodiments of the disclosed methods, the subjects to whom the compositions are administered are chosen from subjects suspected of suffering from at least one disease, disorder, and/or condition associated with a loss of visual acuity, subjects known to be suffering from at least one disease, disorder, and/or condition associated with a loss of visual acuity, and subjects at risk of developing at least one disease, disorder, and/or condition associated with a loss of visual acuity.

In some embodiments, the disclosed methods further comprise identifying a subject as being at risk of developing at least one disease, disorder, and/or condition associated with a loss of visual acuity. In some embodiments, the disclosed methods further comprise identifying a subject as being at risk of developing AMD. In some embodiments, the disclosed methods further comprise identifying a subject as having AMD. In some embodiments, the disclosed methods further comprise identifying a subject as being at risk for AMD progression.

In some embodiments of the methods described herein, the subject is a human. In some embodiments of the methods described herein, the subject is a non-human animal. Non-human animals include mammals, for example, non-human primates, swine, equine, canine, feline, bovine, rodents, and other domestic, farm, and zoo animals.

A subject at risk of developing at least one disease, disorder, and/or condition associated with a loss of visual acuity can be identified by one or more diagnostic or prognostic assays described herein and/or known to those of ordinary skill in the art.

In some embodiments, the methods disclosed herein treat and/or prevent at least one disease, disorder, and/or condition associated with a loss of visual acuity as evidenced by an improvement of visual acuity. In some embodiments, the methods disclosed herein further comprise monitoring the subject for efficacy of administering to the subject a composition as disclosed herein. For example, in some embodiments, the methods disclosed herein further comprise monitoring a subject for improvement of visual acuity which comprises measuring a parameter indicative of visual acuity in the subject at a first time point prior to administration of the composition disclosed herein, measuring the same parameter in the subject at a second time point after administration of the composition disclosed herein, and comparing the two measurements to assess improvement.

The methods disclosed herein comprise administration of a daily dose of a composition disclosed herein. Determining and adjusting an appropriate dosing regimen (e.g., adjusting the number of doses and frequency of dosing) per day can be performed by one of ordinary skill in the relevant art, and will depend upon various factors such as the nature and progression of at least one disease, disorder, and/or condition associated with a loss of visual acuity, and the health and/or age of the subject.

In some embodiments, the composition disclosed herein is formulated in the form of one tablet, two tablets, three tablets, four tablets, etc. and is administered to a subject in a single dose per day, in two doses per day, in three doses per day, in four doses per day, etc. or up to, for example, ten doses per day. In some embodiments, the composition disclosed herein is formulated in the form of four tablets which are administered to a subject in two doses of two tablets each per day. In some embodiments, the composition disclosed herein is administered to a subject for at least one week, at least two weeks, at least three weeks, at least one month, at least 2 months, at least 3 months, at least 4 months, at least 5 months, at least 6 months, at least 12 months, at least one year, or for more than one year.

Also disclosed herein are methods of preventing, stabilizing, reversing and/or treating macular degeneration or visual acuity loss by reducing the risk of developing late stage or advanced age-related macular degeneration in persons with early age-related macular degeneration comprising providing a subject a daily dosage of not less than about; about 500 mg vitamin C, about 400 mg vitamin E, about 80 mg zinc, about 2 mg copper, about 10 mg lutein, about 2 mg zeaxanthin, and about 100 mg coenzyme Q10. In some embodiments, the daily dosage is provided in the form of two tablets taken twice daily or in the form of one tablet taken twice daily.

The effectiveness of the compositions and methods of the present disclosure, for example, in treating and/or preventing at least one disease, disorder, and/or condition associated with a loss of visual acuity, can be determined by a person of ordinary skill in the relevant art. One or any combination of diagnostic methods, including physical examination, assessment and monitoring of symptoms, and performance of analytical tests and methods described herein, may be used for monitoring the at least one disease, disorder, and/or condition. For example, ocular examinations such as biomicroscopy, tonometry, stereoscopic fundus examination (e.g., color fundus photography), macular function assessment, optical coherence tomography (OCT), autofluorescence, and/or angiography (e.g., fluorescein angiography, and OCT based angiography (OCTA)) may be used.

For example, visual acuity can be assessed by any suitable manner known to one of ordinary skill in the art. In some embodiments, visual acuity is assessed by determining the smallest letters the patient can read on a standard vision chart at a set distance. Further for example, progression of AMD can be evaluated by measuring a parameter, such as neovascularization. Maintenance or a reduction in the measured parameter from the first time point to the second time point is indicative of the prevention of AMD progression. In some embodiments, progression of AMD and/or drusen regression is evaluated by determining a particular parameter of drusen in a patient at a first time point (e.g., prior to administration of a high-dose statin), determining the same parameter in the same patient at a second time point (e.g., after administration of a high-dose statin), and comparing the measured parameter at the first time point and the second time point. The parameter of drusen can be, for example, volume, height, diameter, and/or number. For example, drusen regression can be by at least 5%, at least 10% at least 15%, at 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, drusen are reduced 5% to at least 40%, such as 90%. In addition, atrophy can be evaluated, for example, by measuring a parameter such as autofluorescence or retinal thickness and PED can be evaluated for flattening, for example, by measuring a parameter such as volume, height, and/or diameter, and can be evaluated for re-attaching to the Bruch's membrane by, for example, measuring a parameter such as the distance of the separation.

### PROPHETIC EXAMPLES

The following compositions can be prepared. The amounts in each composition are set forth on a daily dosage basis. Each composition can be formulated into two or more dosage units, which dosage units can be administered to a subject daily

| Active Ingredient | Amount (mg) | | | |
|---|---|---|---|---|
| Vitamin C (Ascorbic acid or equivalent) | 500 | 500 | 500 | 500 |
| Vitamin E (alpha tocopherol or equivalent) | 180 | 180 | 180 | 180. |
| Zinc (Zinc oxide or equivalent) | 80 | 25 | 80 | 25 |
| Copper (Copper oxide or equivalent) | 2 | 2 | 2 | 2 |
| Lutein (R′R′) | 10 | 10 | 10 | 10 |
| Zeaxanthin (R′R′) | 2 | 2 | 2 | 2 |
| Coenzyme Q10 | 100 | 100 | 400 | 400 |

Compositions combining the nutritional dietary ingredients listed above are prepared by mixing the recited ingredients. The compositions may be stable for at least two years at about room temperature.

1. A composition for visual and/or heart health comprising:
vitamin C, a prodrug thereof, and/or a pharmaceutically acceptable salt of any of the foregoing;
vitamin E, a prodrug thereof, and/or a pharmaceutically acceptable salt of any of the foregoing;
zinc, a prodrug thereof, and/or a pharmaceutically acceptable salt of any of the foregoing;
copper, a prodrug thereof, and/or a pharmaceutically acceptable salt of any of the foregoing;
lutein, a prodrug thereof, and/or a pharmaceutically acceptable salt of any of the foregoing;
zeaxanthin, a prodrug thereof, and/or a pharmaceutically acceptable salt of any of the foregoing; and
coenzyme Q10, a prodrug thereof, and/or a pharmaceutically acceptable salt of any of the foregoing.

2. The composition of claim 1, wherein the vitamin C, a prodrug thereof, and/or a pharmaceutically acceptable salt of any of the foregoing comprises ascorbic acid.

3. The composition of claim 1, wherein the vitamin E, a prodrug thereof, and/or a pharmaceutically acceptable salt of any of the foregoing comprises alpha-tocopherol.

4. The composition of claim 1, wherein the zinc, a prodrug thereof, and/or a pharmaceutically acceptable salt of any of the foregoing comprises zinc oxide.

5. The composition of claim 1, wherein the copper, a prodrug thereof, and/or a pharmaceutically acceptable salt of any of the foregoing comprises copper oxide.

6. The composition of claim 1, wherein the lutein, a prodrug thereof, and/or a pharmaceutically acceptable salt of any of the foregoing comprises lutein (R′,R′).

7. The composition of claim 1, wherein the zeaxanthin, a prodrug thereof, and/or a pharmaceutically acceptable salt of any of the foregoing comprises zeaxanthin (R′,R′).

8. The composition of claim 1, wherein the zeaxanthin, a prodrug thereof, and/or a pharmaceutically acceptable salt of any of the foregoing comprises zeaxanthin (R′,S′).

9. The composition of claim 1, wherein the coenzyme Q10, a prodrug thereof, and/or a pharmaceutically acceptable salt of any of the foregoing comprises ubiquinol.

10. The composition of claim 1, wherein the coenzyme Q10, a prodrug thereof, and/or a pharmaceutically acceptable salt of any of the foregoing comprises ubiquinone.

11-20. (canceled)

21. The composition of claim 1, wherein the composition comprises about 350 mg to about 800 mg of vitamin C or an equivalent amount of a prodrug, a pharmaceutically acceptable salt, and/or a pharmaceutically acceptable salt of a prodrug of vitamin C.

22. The composition of claim 1, wherein the composition comprises about 100 mg to about 550 mg of vitamin E or an equivalent amount of a prodrug, a pharmaceutically acceptable salt, and/or a pharmaceutically acceptable salt of a prodrug of vitamin E.

23. The composition of claim 1, wherein the composition comprises about 20 mg to about 90 mg of zinc or an equivalent amount of a prodrug, a pharmaceutically acceptable form, and/or a pharmaceutically acceptable salt of a prodrug of zinc.

24. The composition of claim 1, wherein the composition comprises about 1.5 mg to about 2.5 mg of copper or an equivalent amount of a prodrug, a pharmaceutically acceptable form, and/or a pharmaceutically acceptable salt of a prodrug of copper.

25. The composition of claim 1, wherein the composition comprises about 5 mg to about 50 mg of lutein or an equivalent amount of a prodrug, a pharmaceutically acceptable salt, and/or a pharmaceutically acceptable salt of a prodrug of lutein.

26. The composition of claim 1, wherein the composition comprises about 1.0 mg to about 20.0 mg of zeaxanthin or an equivalent amount of a prodrug, a pharmaceutically acceptable salt, and/or a pharmaceutically acceptable salt of a prodrug of zeaxanthin.

27. The composition of claim 1, wherein the composition comprises about 1.0 mg to about 20.0 mg of R′,R′-zeaxanthin or an equivalent amount of a prodrug, a pharmaceutically acceptable salt, and/or a pharmaceutically acceptable salt of a prodrug of R′,R′-zeaxanthin.

28. The composition of claim 1, wherein the composition comprises about 1.0 mg to about 20.0 mg of R′,S′-zeaxanthin or an equivalent amount of a prodrug, a pharmaceutically acceptable salt, and/or a pharmaceutically acceptable salt of a prodrug of R′,S′-zeaxanthin.

29. The composition of claim 1, wherein the composition comprises about 1.0 mg to about 20.0 mg of a mixture of R′,R′-zeaxanthin and R′,S′-zeaxanthin, or an equivalent amount of prodrugs, pharmaceutically acceptable salts, and/or a pharmaceutically acceptable salts of prodrugs of R′,R′-zeaxanthin and R′,S′-zeaxanthin.

30. The composition of claim 1, wherein the composition comprises about 50 mg to about 500 mg of coenzyme Q10 or an equivalent amount of a prodrug, a pharmaceutically acceptable salt, and/or a pharmaceutically acceptable salt of a prodrug of coenzyme Q10.

31-38. (canceled)

39. The composition of claim 1, wherein the composition comprises: about 500 mg vitamin C, optionally in the form of ascorbic acid; about 400 mg vitamin E, optionally in the form of dl-alpha tocopheryl acetate; about 80 mg zinc, optionally in the form of zinc oxide; about 2 mg copper, optionally in the form of copper oxide; about 10 mg R′,R′ lutein; about 2 mg R′,R′ zeaxanthin; and about 100 mg coenzyme Q10.

40. The composition of claim 1, wherein the composition comprises: about 500 mg vitamin C, optionally in the form of ascorbic acid; about 400 mg vitamin E, optionally in the form of dl-alpha tocopheryl acetate; about 25 mg zinc, optionally in the form of zinc oxide; about 2 mg copper, optionally in the form of copper oxide; about 10 mg R′,R′ lutein; about 2 mg R′,R′ zeaxanthin; about 100 mg coenzyme Q10.

41. The composition of claim 1, wherein the composition comprises: about 500 mg vitamin C, optionally in the form of ascorbic acid; about 180 mg vitamin E, optionally in the form of dl-alpha tocopheryl acetate; about 80 mg zinc, optionally in the form of zinc oxide; about 2 mg copper, optionally in the form of copper oxide; about 10 mg R′,R′ lutein; about 2 mg R′,R′ zeaxanthin; and about 100 mg coenzyme Q10.

42. The composition claim 1, wherein the composition comprises: about 500 mg vitamin C, optionally in the form of ascorbic acid; about 180 mg vitamin E, optionally in the form of dl-alpha tocopheryl acetate; about 25 mg zinc, optionally in the form of zinc oxide; about 2 mg copper, optionally in the form of copper oxide; about 10 mg R′,R′ lutein; about 2 mg R′,R′ zeaxanthin; and about 100 mg coenzyme Q10.

43. The composition of claim 1, wherein the composition comprises: about 500 mg vitamin C, optionally in the form of ascorbic acid; about 400 mg vitamin E, optionally in the form of dl-alpha tocopheryl acetate; about 80 mg zinc, optionally in the form of zinc oxide; about 2 mg copper, optionally in the form of copper oxide; about 10 mg R′,R′ lutein; about 2 mg R′,S′ zeaxanthin; and about 100 mg coenzyme Q10.

44. The composition of claim 1, wherein the composition comprises: about 500 mg vitamin C, optionally in the form of ascorbic acid; about 400 mg vitamin E, optionally in the form of dl-alpha tocopheryl acetate; about 25 mg zinc, optionally in the form of zinc oxide; about 2 mg copper, optionally in the form of copper oxide; about 10 mg R′,R′ lutein; about 2 mg R′,S′ zeaxanthin; about 100 mg coenzyme Q10.

45. The composition of claim 1, wherein the composition comprises: about 500 mg vitamin C, optionally in the form of ascorbic acid; about 180 mg vitamin E, optionally in the form of dl-alpha tocopheryl acetate; about 80 mg zinc, optionally in the form of zinc oxide; about 2 mg copper, optionally in the form of copper oxide; about 10 mg R′,R′ lutein; about 2 mg R′,S′ zeaxanthin; and about 100 mg coenzyme Q10.

46. The composition of claim 1, wherein the composition comprises: about 500 mg vitamin C, optionally in the form of ascorbic acid; about 180 mg vitamin E, optionally in the form of dl-alpha tocopheryl acetate; about 25 mg zinc, optionally in the form of zinc oxide; about 2 mg copper, optionally in the form of copper oxide; about 10 mg R′,R′ lutein; about 2 mg R′,S′ zeaxanthin; and about 100 mg coenzyme Q10.

47. The composition of claim 1, wherein the composition comprises: about 500 mg vitamin C, optionally in the form of ascorbic acid; about 400 mg vitamin E, optionally in the form of dl-alpha tocopheryl acetate; about 80 mg zinc, optionally in the form of zinc oxide; about 2 mg copper, optionally in the form of copper oxide; about 10 mg R′,R′ lutein; about 2 mg R′,R′ zeaxanthin; and about 400 mg coenzyme Q10.

48. The composition of claim 1, wherein the composition comprises: about 500 mg vitamin C, optionally in the form of ascorbic acid; about 400 mg vitamin E, optionally in the form of dl-alpha tocopheryl acetate; about 25 mg zinc, optionally in the form of zinc oxide; about 2 mg copper, optionally in the form of copper oxide; about 10 mg R′,R′ lutein; about 2 mg R′,R′ zeaxanthin; about 400 mg coenzyme Q10.

49. The composition of claim 1, wherein the composition comprises: about 500 mg vitamin C, optionally in the form of ascorbic acid; about 180 mg vitamin E, optionally in the form of dl-alpha tocopheryl acetate; about 80 mg zinc, optionally in the form of zinc oxide; about 2 mg copper, optionally in the form of copper oxide; about 10 mg R′,R′ lutein; about 2 mg R′,R′ zeaxanthin; and about 400 mg coenzyme Q10.

50. The composition of claim 1, wherein the composition comprises: about 500 mg vitamin C, optionally in the form of ascorbic acid; about 180 mg vitamin E, optionally in the form of dl-alpha tocopheryl acetate; about 25 mg zinc, optionally in the form of zinc oxide; about 2 mg copper, optionally in the form of copper oxide; about 10 mg R′,R′ lutein; about 2 mg R′,R′ zeaxanthin; and about 400 mg coenzyme Q10.

51. The composition of claim 1, wherein the composition is in the form of one or more tablets, capsules, soft gels, liquid, or powder.

52. The composition of claim 1, wherein the composition is in the form of an immediate release formulation or a modified release formulation.

53. The composition of claim 1, wherein the composition is formulated into one, two, three, or four dosage units for daily administration.

54. The composition of claim 1, wherein the composition is formulated into one, two, three, or four tablets for daily administration.

55. The composition of claim 1, wherein at least one vitamin is from a natural source of said vitamin.

56. The composition of claim 1, wherein at least one vitamin is from a synthetic source of said vitamin.

57. (canceled)

58. The composition of claim 56, wherein the composition comprises about 350 mg to about 800 mg of vitamin C or an equivalent amount of a prodrug, a pharmaceutically acceptable salt, and/or a pharmaceutically acceptable salt of a prodrug of vitamin C; about 100 mg to about 550 mg of vitamin E or an equivalent amount of a prodrug, a pharmaceutically acceptable salt, and/or a pharmaceutically acceptable salt of a prodrug of vitamin E; about 20 mg to about 90 mg of zinc or an equivalent amount of a prodrug, a pharmaceutically acceptable form, and/or a pharmaceutically acceptable salt of a prodrug of zinc; about 1.5 mg to about 2.5 mg of copper or an equivalent amount of a prodrug, a pharmaceutically acceptable form, and/or a pharmaceutically acceptable salt of a prodrug of copper; about 5 mg to about 50 mg of lutein or an equivalent amount of a prodrug, a pharmaceutically acceptable salt, and/or a pharmaceutically acceptable salt of a prodrug of lutein; about 1 mg to about 20 mg of zeaxanthin or an equivalent amount of a prodrug, a pharmaceutically acceptable salt, and/or a pharmaceutically acceptable salt of a prodrug of zeaxanthin; and about 50 mg to about 500 mg of coenzyme Q10 or an equivalent amount of a prodrug, a pharmaceutically acceptable salt, and/or a pharmaceutically acceptable salt of a prodrug of coenzyme Q10.

59. The composition of claim 1, wherein the composition further comprises B vitamins.

60. The composition of claim 1, wherein the composition further comprises omega-3 fatty acids.

61. The composition of claim 1, wherein the composition further comprises very long chain polyunsaturated fatty acids.

62. The composition of claim 1, wherein the composition is on a daily dosage basis.

63-65. (canceled)

66. A method of delaying the progression and/or onset of macular degeneration, the method comprising administering to a person in need thereof the composition of claim 1.

67. A method for treating and/or preventing at least one disease, disorder, and/or condition associated with a loss of visual acuity, the method comprising administering to a subject the composition of claim 1.

68. A method of delaying the progression and/or onset of heart failure, the method comprising administering to a person in need thereof the composition of claim 1.

69. (canceled)

70. A method for treating at least one eye disease, disorder, and/or condition and at least one heart disease, disorder, and/or condition, the method comprising administering to a subject the composition of claim 1.

71. The method of claim 70, wherein the method further comprises lowering blood pressure.

72. A method for lowering blood pressure, the method comprising administering to a subject the composition of claim 1.

73. (canceled)
